SEQUENCE LISTING (6 sequences)

SEQ ID NO: 1 - protein, 28 aa, Vasoactive Intestinal Peptide
  AMIDATION 28
HSDAVFTDNYTRLRKQMAVKKYLNSILN

SEQ ID NO: 2 - protein, 29 aa, Growth Hormone Releasing Hormone
  AMIDATION 29
YADAIFTNSYRKVLGQLSARKLLQDIMSR

SEQ ID NO: 3 - protein, 27 aa, PACAP27
  AMIDATION 27
HSDGIFTDSYSRYRKQMAVKKYLAAVL

SEQ ID NO: 4 - protein, 28 aa, R2P3
  ACETYLATION 1
  VARIANT 2: D-Phenylalanine
HFDAVFTNSYRKVLKRLSARKLLQDILC

SEQ ID NO: 5 - protein, 28 aa, R2P11
  ACETYLATION 1
  VARIANT 2: D-Phenylalanine
HFDAVFTNSYRKVLKRLSVRKLLQDILC

SEQ ID NO: 6 - protein, 27 aa, R2P16
  ACETYLATION 1
  VARIANT 2: D-Phenylalanine
HFDAVFTNSYRKVLKRLSARKLLQSIL

(19) United States
(12) Patent Application Publication
(10) Pub. No.: US 2005/0203009 A1
(43) Pub. Date: Sep. 15, 2005

(54) VPAC1 selective antagonists and their pharmacological methods of use

(75) Inventors: Clark Pan, Castro Valley, CA (US); Steve Roczniak, Lafayette, CA (US)

Correspondence Address: John W. Mahoney;Bayer Pharmaceuticals Corporation, 800 Dwight Way, P.O. Box 1986, Berkeley, CA, 94701-1986, US

(73) Assignee: Bayer Pharmaceuticals Corporation

(21) Appl. No.: 10/799,897

(22) Filed: Mar. 12, 2004

(51) Int. Cl. A61K 38/17; C07K 14/71; C07H 21/04; C12N 15/09

(52) U.S. Cl. 514/12; 530/350; 536/23.5; 435/69.1; 435/320.1; 435/325

(57) ABSTRACT

The disclosed invention relates to selective VPAC1 antagonists, related formulations, dosages and methods of use. The selective VPAC1 antagonists of the invention comprise a vasoactive intestinal peptide component and a growth hormone releasing hormone component capable of selectively binding to and antagonizing the VPAC1 receptor at significantly lower concentrations than those concentrations at which it binds to and antagonizes the VPAC2 receptor.

This invention relates to a VPAC1 selective antagonist. In addition, related formulations, dosages and methods of administration thereof for therapeutic purposes are provided. These selective VPAC1 selective antagonists and associated compositions and methods are useful in providing a treatment option for individuals afflicted with various forms of cancer.

### BACKGROUND

Pituitary adenylate cyclase-activating polypeptide (PACAP) belongs to the secretin/glucagon/vasoactive intestinal peptide (VIP) family of peptides (Sherwood, N. M., Krueckl, S. L., and McRory, J. E. (2000) Endocr Rev 21, 619-70). These peptides are expressed as fragments of larger proteins that are processed by proteolysis followed by C-terminal amidation to generate the mature amidated peptides. PACAP exists as a 38-residue form (PACAP38), and as a shorter form corresponding to the N-terminal 27 amino acids of PACAP38 (PACAP27). Both forms of PACAP bind to and activate the G-protein-coupled receptors PAC1, VPAC1, and VPAC2, whereas the related 28mer peptide VIP only recognizes VPAC1 and VPAC2 (Laburthe, M., and Couvineau, A. (2002) Regul Pept 108, 165).

The VPAC1 receptor is an attractive cancer therapy target for 3 reasons: 1) It is over-expressed in a vast majority of human cancers. 2) VPAC1 expression levels have been found to greatly exceed those of VPAC2 and PAC1 in tumors. 3) Binding of VIP to the VPAC1 has been shown to promote cell proliferation (Moody, T. W., Hill, J. M., and Jensen, R. T. (2003) Peptides 24, 163-77 and Moody, T. W., Leyton, J., Coelho, T., Jakowlew, S., Takahashi, K., Jameison, F., Koh, M., Fridkin, M., Gozes, I., and Knight, M. (1997) Life Sci 61, 1657-66). As a result, treatment of cancer patients with a VPAC1 antagonist should result in decreased growth of human tumors. Indeed, in a PC-3 tumor xenograft model, mice treated with the non-selective VPAC1/VPAC2 antagonist JV-1-53 (Rekasi, Z., Varga, J. L., Schally, A. V., Halmos, G., Groot, K., and Czompoly, T. (2000) Proc Natl Acad Sci USA 97, 1218-23) had reduced tumor volume and weight compared to control mice (Plonowski, A., Varga, J. L., Schally, A. V., Krupa, M., Groot, K., and Halmos, G. (2002) Int J Cancer 98, 624-9). Likewise, the broad-spectrum PAC1/VPAC1/VPAC2 antagonist VIPhybrid (Moody, T. W., Jensen, R. T., Fridkin, M., and Gozes, I. (2002) J Mol Neurosci 18, 29-35) inhibits non-small cell lung cancer (Moody, T. W., Zia, F., Draoui, M., Brenneman, D. E., Fridkin, M., Davidson, A., and Gozes, I. (1993) Proc Natl Acad Sci USA 90, 4345-9), breast cancer (Zia, H., Hida, T., Jakowlew, S., Birrer, M., Gozes, Y., Reubi, J. C., Fridkin, M., Gozes, I., and Moody, T. W. (1996) Cancer Res 56, 3486-9), and pancreatic tumor growth (Zia, H., Leyton, J., Casibang, M., Hau, V., Brenneman, D., Fridkin, M., Gozes, I., and Moody, T. W. (2000) Life Sci 66, 379-87) in vivo. Furthermore, an affinity-improved analog of VIPhybrid enhances the anti-proliferation effect of chemotherapeutic agents on cancer cell lines (Moody, T. W., Leyton, J., Chan, D., Brenneman, D. C., Fridkin, M., Gelber, E., Levy, A., and Gozes, I. (2001) Breast Cancer Res Treat 68, 55-64 and Gelber, E., Granoth, R., Fridkin, M., Dreznik, Z., Brenneman, D. E., Moody, T. W., and Gozes, I. (2001) Cancer 92, 2172-80).

Although these non-selective peptide antagonists of PACAP and VIP receptors may demonstrate excellent anti-cancer properties, they are not ideal drug candidates due to the possible side effects associated with non-discriminate receptor inhibition. Clinical applications will, however, require selective modulation of the VPAC1 to minimize potential side effects mediated by other receptors because PACAP and VIP have broad physiological effects on the nervous, endocrine, cardiovascular, reproductive, muscular, and immune systems (4). PG 97-269, a VIP/growth hormone releasing hormone (GHRH) hybrid, is a VPAC1 selective antagonist (Gourlet, P., De Neef, P., Cnudde, J., Waelbroeck, M., and Robberecht, P. (1997) Peptides 18, 1555-60) and, while it is a highly selective binder of human VPAC1, a more potent inhibitor of VPAC1 activity would have greater therapeutic utility. In addition, PG 97-269 has numerous mutations relative to the native peptides VIP and GHRH, which may lead to an undesired immunogenic response. Furthermore, because of its small size, PG 97-269 will likely have a short in vivo duration of action.

We have developed a recombinant VPAC1 selective antagonist derived from a human VIP/GHRH mutated at several amino acid residues. This receptor antagonist selectively binds with high affinity to the human VPAC1 and, in cell-based assays, inhibits VPAC1-mediated activity including H727 cell proliferation. In addition, we have developed a method of site-specifically conjugating the mutein with a polymer such as polyethylene glycol (PEG) as a means of potentially enhancing the pharmacokinetic profile of the mutein while retaining its receptor selectivity.

### SUMMARY OF THE INVENTION

The invention provides reagents and methods of inhibiting VPAC1-mediated tumorigenesis. This and other objects of the invention are provided by one or more of the embodiments listed below.

In one embodiment, the invention provides a purified preparation of a VPAC1 selective antagonist comprising an amino acid sequence as set forth in SEQ ID NO: 4, SEQ ID NO: 5, SEQ ID NO: 6.

In one embodiment, the modified VPAC1 selective antagonist of the invention inhibits PACAP27 binding to the VPAC1 preferably with an IC50 of about 0.1 nM to about 10 μM, more preferably with an IC50 of about 0.5 nM to about 1 μM, or most preferably with an IC50 of about 1.0 nM to about 100 nM.

In another embodiment, a modified VPAC1 selective antagonist of the invention inhibits VPAC1 mediated activity with at least 100-fold selectivity for VPAC1 over VPAC2.

In another embodiment, the modified VPAC1 selective antagonist of the invention inhibits the cAMP induction by VIP in VPAC1 expressing cells preferably with an IC50 of about 0.1 nM to about 10 μM, more preferably with an IC50 of about 0.5 nM to about 1 μM, or most preferably with an IC50 of about 1.0 nM to about 100 nM.

In still another embodiment, the VPAC1 selective antagonist of the invention inhibits the proliferative response of H727 tumor cells with an IC50 of about 0.1 nM to about 10 μM, more preferably with an IC50 of about 0.5 nM to about 1 μM, or most preferably with an IC50 of about 1.0 nM to about 100 nM.

In another embodiment, the VPAC1 selective antagonist of the invention can be coupled to a non-protein polymer at the C-terminal amino acid residue. In one aspect of this embodiment, the C-terminal amino acid residue is cysteine.

In still another embodiment, the VPAC1 selective antagonist of the invention, when coupled to a non-protein polymer has a plasma half-life which is at least about 2-10 fold greater than that of an unmodified VPAC1 selective antagonist.

The invention also provides pharmaceutical compositions comprising: (a) a VPAC1 selective antagonist which binds to the human VPAC1; and (b) a pharmaceutically acceptable carrier.

The invention also provides methods for treating a human disorder associated with increased expression and activity of the VPAC1, comprising the steps of: (a) providing a human having a condition in which activity of VPAC1 is increased; and (b) administering to said human an effective amount of VPAC1 selective antagonist of the invention or a pharmaceutical composition of the invention. In one aspect, the disorder is cancer or related conditions.

Specific preferred embodiments of the present invention will become evident from the following more detailed description of certain preferred embodiments and the claims.

### DETAILED DESCRIPTION OF THE INVENTION

This invention relates to a selective VPAC1 selective antagonist. In addition, related formulations, dosages and methods of administration thereof for therapeutic purposes are provided. These selective VPAC1 selective antagonists and associated compositions and methods are useful in providing a treatment option for individuals afflicted with various forms of cancer.

Unless otherwise required by context, singular terms shall include pluralities and plural terms shall include the singular.

The section headings used herein are for organizational purposes only and are not to be construed as limiting the subject matter described. All references cited in this application are expressly incorporated by reference herein.

As used herein, the term “VPAC1 selective antagonist” refers to a compound that is able to selectively bind to VPAC1 and reduce VPAC1 activation by an agonist particularly vasoactive intestinal peptide (VIP). VPAC1 selective antagonists will bind to the VPAC1 at significantly lower concentrations than the VPAC2 receptor. Selectivity is determined by comparing the IC50's of the receptor antagonist for the VPAC1 and VPAC2 receptors. Typically, the selectivity for the VPAC1 will be at least about 2:1, preferably at least about 10:1, more preferably at least about 100:1 and most preferably at least 1000:1 over the VPAC2 receptor. The lower the IC50 of a receptor antagonist relative to its IC50 for other receptors, the greater the selectivity.

As used herein, the term “hybrid” means a protein comprised of different protein domains, forming a functional, chimeric protein with the characteristics of the individual domains.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., Molecular Cloning, A Laboratory Manual (Cold Spring Harbor Laboratories, 1989); Davis et al., Basic Methods in Molecular Biology (Elsevier, 1986); and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, may be maintained transiently as an episomal element without being replicated, or may replicate independently as a plasmid. A cell is considered to have been stably transformed when the DNA is replicated with the division of the cell.

The term “identity,” as known in the art, refers to a relationship between the sequences of two or more polypeptide molecules or two or more nucleic acid molecules, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”).

The term “similarity” is a related concept, but in contrast to “identity,” “similarity” refers to a measure of relatedness which includes both identical matches and conservative substitution matches. If two polypeptide sequences have, for example, 10/20 identical amino acids, and the remainder are all non-conservative substitutions, then the percent identity and similarity would both be 50%. If in the same example, there are five more positions where there are conservative substitutions, then the percent identity remains 50%, but the percent similarity would be 75% (15/20). Therefore, in cases where there are conservative substitutions, the percent similarity between two polypeptides will be higher than the percent identity between those two polypeptides.

Identity and similarity of related polypeptides can be readily calculated by known methods. Such methods include, but are not limited to, those described in COMPUTATIONAL MOLECULAR BIOLOGY, (Lesk, A. M., ed.), 1988, Oxford University Press, New York; BIOCOMPUTING: INFORMATICS AND GENOME PROJECTS, (Smith, D. W., ed.), 1993, Academic Press, New York; COMPUTER ANALYSIS OF SEQUENCE DATA, Part 1, (Griffin, A. M., and Griffin, H. G., eds.), 1994, Humana Press, New Jersey; von Heinje, G., SEQUENCE ANALYSIS IN MOLECULAR BIOLOGY, 1987, Academic Press; SEQUENCE ANALYSIS PRIMER, (Gribskov, M. and Devereux, J., eds.), 1991, M. Stockton Press, New York; Carillo et al., 1988, SIAM J. Applied Math., 48:1073; and Durbin et al., 1998, BIOLOGICAL SEQUENCE ANALYSIS, Cambridge University Press.

Preferred methods to determine identity are designed to give the largest match between the sequences tested. Methods to determine identity are described in publicly available computer programs. Preferred computer program methods to determine identity between two sequences include, but are not limited to, the GCG program package, including GAP (Devereux et al., 1984, Nucl. Acid. Res., 12:387; Genetics Computer Group, University of Wisconsin, Madison, Wis.), BLASTP, and FASTA (Altschul et al., 1990, J. Mol. Biol., 215:403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990, supra). The well-known Smith Waterman algorithm may also be used to determine identity.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). In certain embodiments, a gap opening penalty (which is calculated as three-times the average diagonal; where the “average diagonal” is the average of the diagonal of the comparison matrix being used; the “diagonal” is the score or number assigned to each perfect amino acid match by the particular comparison matrix) and a gap extension penalty (which is usually one-tenth of the gap opening penalty), as well as a comparison matrix such as PAM250 or BLOSUM 62 are used in conjunction with the algorithm. In certain embodiments, a standard comparison matrix (see Dayhoff et al., 1978, Atlas of Protein Sequence and Structure, 5:345-352 for the PAM 250 comparison matrix; Henikoffet al., 1992, Proc. Natl. Acad. Sci USA, 89:10915-10919 for the BLOSUM 62 comparison matrix) is also used by the algorithm.

In certain embodiments, the parameters for a polypeptide sequence comparison include the following:
Algorithm: Needleman et al., 1970, J. Mol. Biol., 48:443-453;
Comparison matrix: BLOSUM 62 from Henikoffet al., 1992, supra;
Gap Penalty: 12
Gap Length Penalty: 4
Threshold of Similarity: 0

The GAP program may be useful with the above parameters. In certain embodiments, the aforementioned parameters are the default parameters for polypeptide comparisons (along with no penalty for end gaps) using the GAP algorithm.

As used herein, the twenty conventional amino acids and their abbreviations follow conventional usage. See IMMUNOLOGY—A SYNTHESIS, 2nd Edition, (E. S. Golub and D. R. Gren, Eds.), Sinauer Associates: Sunderland, Mass., 1991, incorporated herein by reference for any purpose. Stereoisomers (e.g., d-amino acids) of the twenty conventional amino acids; unnatural amino acids such as α, α-disubstituted amino acids, N-alkyl amino acids, lactic acid, and other unconventional amino acids may also be suitable components for polypeptides of the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, (σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Naturally occurring residues may be divided into classes based on common side chain properties:
1) hydrophobic: norleucine (Nor), Met, Ala, Val, Leu, Ile;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Trp, Tyr, Phe.

Conservative amino acid substitutions may involve exchange of a member of one of these classes with another member of the same class. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

Non-conservative substitutions may involve the exchange of a member of one of these classes for a member from another class. Such substituted residues may be introduced into regions of a human protein that are homologous with non-human proteins, or into the non-homologous regions of the molecule.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine −3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included. One may also identify epitopes from primary amino acid sequences on the basis of hydrophilicity. These regions are also referred to as “epitopic core regions.”

Amino acid substitutions that exemplify the concepts presented above are set forth in Table 1.

TABLE 1
Amino Acid Substitutions

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, | Leu |
| | Norleucine | |
| Leu | Norleucine, Ile, Val, Met, Ala, | Ile |
| | Phe | |
| Lys | Arg, 1,4 Diamino-butyric Acid, | Arg |
| | Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, | Leu |
| | Norleucine | |

A skilled artisan will be able to determine suitable variants of the polypeptide as set forth herein using well-known techniques. In certain embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three dimensional structure. In certain embodiments, one skilled in the art may choose to not make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult, 1996, Curr. Op. in Biotech. 7:422-427; Chou et al., 1974, Biochemistry 13:222-245; Chou et al., 1974, Biochemistry 113:211-222; Chou et al., 1978, Adv. Enzymol. Relat. Areas Mol. Biol. 47:45-148; Chou et al., 1979, Ann. Rev. Biochem. 47:251-276; and Chou et al., 1979, Biophys. J. 26:367-384. Moreover, computer programs are currently available to assist with predicting secondary structure. One method of predicting secondary structure is based upon homology modeling. For example, two polypeptides or proteins that have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., 1999, Nucl. Acid. Res. 27:244-247. It has been suggested (Brenner et al., 1997, Curr. Op. Struct. Biol. 7:369-376) that there are a limited number of folds in a given polypeptide or protein and that once a critical number of structures have been resolved, structural prediction will become dramatically more accurate.

Additional methods of predicting secondary structure include “threading” (Jones, 1997, Curr. Opin. Struct. Biol. 7:377-87; Sippl et al., 1996, Structure 4:15-19), “profile analysis” (Bowie et al., 1991, Science 253:164-170; Gribskov et al., 1990, Meth. Enzym. 183:146-159; Gribskov et al., 1987, Proc. Nat. Acad. Sci. 84:4355-4358), and “evolutionary linkage” (See Holm, 1999, supra; and Brenner, 1997, supra).

Additional preferred variants include cysteine variants wherein one or more cysteine residues are deleted from or substituted for another amino acid (e.g., serine) compared to the parent amino acid sequence. Cysteine variants may be useful when proteins must be refolded into a biologically active conformation such as after the isolation of insoluble inclusion bodies. Cysteine variants generally have fewer cysteine residues than the native protein, and typically have an even number to minimize interactions resulting from unpaired cysteines.

In additional embodiments, protein variants can include mutations such as substitutions, additions, deletions, or any combination thereof, and are typically produced by site-directed mutagenesis using one or more mutagenic oligonucleotide(s) according to methods described herein, as well as according to methods known in the art (see, for example, Sambrook et al., MOLECULAR CLONING: A LABORATORY MANUAL, 3rd Ed., 2001, Cold Spring Harbor, N.Y. and Berger and Kimmel, METHODS IN ENZYMOLOGY, Volume 152, Guide to Molecular Cloning Techniques, 1987, Academic Press, Inc., San Diego, Calif., which are incorporated herein by reference).

According to certain embodiments, amino acid substitutions are those that: (1) reduce susceptibility to proteolysis, (2) reduce susceptibility to oxidation, (3) alter binding affinity for forming protein complexes, (4) alter binding affinities, and/or (5) confer or modify other physicochemical or functional properties on such polypeptides. According to certain embodiments, single or multiple amino acid substitutions (in certain embodiments, conservative amino acid substitutions) may be made in the naturally occurring sequence (in certain embodiments, in the portion of the polypeptide outside the domain(s) forming intermolecular contacts). In preferred embodiments, a conservative amino acid substitution typically does not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in PROTEINS, STRUCTURES AND MOLECULAR PRINCIPLES, (Creighton, Ed.), 1984, W. H. Freeman and Company, New York; INTRODUCTION TO PROTEIN STRUCTURE (C. Branden and J. Tooze, eds.), 1991, Garland Publishing, New York, N.Y.; and Thornton et al., 1991, Nature 354:105, each of which are incorporated herein by reference.

Peptide analogs are commonly used in the pharmaceutical industry as non-peptide drugs with properties analogous to those of the template peptide. These types of non-peptide compound are termed “peptide mimetics” or “peptidomimetics”. See Fauchere, 1986, Adv. Drug Res. 15:29; Veber & Freidinger, 1985, TINS p.392; and Evans et al,. 1987, J. Med. Chem. 30:1229, which are incorporated herein by reference for any purpose. Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH2-NH—, —CH2-S—, —CH2-CH2-, —CH═CH-(cis and trans), —COCH2-, —CH(OH)CH2-, and —CH2SO—, by methods well known in the art. Systematic substitution of one or more amino acids of a consensus sequence with a d-amino acid of the same type (e.g., d-lysine in place of 1-lysine) may be used in certain embodiments to generate more stable peptides. In addition, constrained peptides comprising a consensus sequence or a substantially identical consensus sequence variation may be generated by methods known in the art (Rizo & Gierasch, 1992, Ann. Rev. Biochem. 61:387, incorporated herein by reference for any purpose); for example, by adding internal cysteine residues capable of forming intramolecular disulfide bridges which cyclize the peptide.

### Characteristics of VPAC1 Selective Antogonist

The VPAC1 selective antagonists of the invention are based on a framework derived from VIP and GHRH sequences preceded by acylated histidine and D-phenylalanine residues at positions 1 and 2, respectively. Subsequent C-terminal residues consist of variegated VIP/GHRH hybrid sequences including site-specifically mutagenized residues as disclosed in SEQ ID NOS 4-6. Table 2 provides the sequence listings of SEQ ID NOS 1-6.

VPAC1 selective antagonists of the invention also includes the peptides described above with additional amino acid substitutions wherein said substitutions enable the site-specific coupling of at least one non-protein polymer, such as polypropylene glycol, polyoxyalkylene, or polyethylene glycol (PEG) molecule to the mutein. Site-specific coupling of PEG, for example, allows the generation of a modified mutein which possesses the benefits of a polyethylene-glycosylated (PEGylated) molecule, namely increased plasma half life and decreased immunogenicity while maintaining greater potency over non-specific PEGylation strategies such as N-terminal and lysine side-chain PEGylation. Such modified VPAC1 receptor antagonists bind the VPAC1 with an affinity loss not greater than 10-fold relative to that of unmodified VPAC1 selective antagonists. Modified VPAC1 selective antagonists inhibit VPAC1-mediated activity with a loss of potency not greater than 10-fold relative to that of unmodified VPAC1 selective antagonists. In addition, modified VPAC1 selective antagonists possess a plasma half-life which is at least 2 to 10-fold greater than that of unmodified VPAC1 antagonists.

The VPAC1 selective antagonists of the invention may also be characterized by amino acid insertions, deletions, substitutions and modifications at one or more sites in or at the other residues of the native VIP polypeptide chain. In accordance with this invention any such insertions, deletions, substitutions and modifications should maintain the VPAC1 antagonist activity of the peptide.

The IC50 of the VPAC1 selective antagonist of the present invention can be assayed using any method known in the art, including protocols such the receptor competition assay outlined in Example 4. This assay measures the ability of an antagonist to selectively inhibit binding of a radio-labeled VPAC1 ligand.

The capacity of the VPAC1 selective antagonist of the present invention to inhibit the proliferative response of cancer cells can be assessed using proliferative assays as outlined in Example 6 and this capacity expressed as an Inhibitory Concentration 50% (IC50).

In the receptor competition assay of Example 4, VPAC1 selective antagonists of the present invention specifically inhibit VPAC1 activity with a preferred IC50 in the range of from about 1.0 nM to about 100 nM. More preferred embodiments of the present invention inhibit VPAC1 with an IC50 of approximately 0.5 nM to about 1.0 μM. Still more preferred embodiments of the present invention inhibit VPAC1 with an IC50 of approximately 0.1 nM to about 10 μM. Additionally, human VPAC1 selective antagonists of the present invention, as envisioned, will bind to the human VPAC1 and neutralize its capacity to promote cancer cell proliferation with a preferred IC50 ranging from about 1.0 nM to about 100 nM.

More preferred embodiments of VPAC1 selective antagonists of the present invention provides a preparation wherein the VPAC1 selective antagonists are coupled to a non-protein polymer selected from a group consisting of polyethylene glycol, polypropylene glycol and polyoxyalkenes and exhibit a plasma half-life that is preferably at least 2 to 10-fold greater than that of an unmodified VPAC1 selective antagonists. The most preferred embodiments of the present invention will exhibit a plasma half-life which is 10-100-fold greater than that of unmodified VPAC1 selective antagonists. In one aspect of this embodiment, the VPAC1 selective antagonist of the invention is comprised of the polypeptide sequence set forth in SEQ ID NOS 5 and 6.

Number of VPAC1 selective antagonists with the characteristics described above have been identified by screening candidates with the above assays. The embodiments of the present invention have the polypeptide sequences shown in Table 2 (SEQ ID NOS 4-6).

Peptides as provided by the invention can be advantageously synthesized by any of the chemical synthesis techniques known in the art, particularly solid-phase synthesis techniques, for example, using commercially-available automated peptide synthesizers. The mimetics of the present invention can be synthesized by solid phase or solution phase methods conventionally used for the synthesis of peptides (see, for example, Merrifield, 1963, J. Amer. Chem. Soc. 85: 2149-54; Carpino, 1973, Acc. Chem. Res. 6: 191-98; Birr, 1978, Aspects of the Merrifield Peptide Synthesis, Springer-Verlag: Heidelberg; The Peptides: Analysis, Synthesis, Biology, Vols. 1, 2, 3, 5, (Gross & Meinhofer, eds.), Academic Press: New York, 1979; Stewart et al., 1984, Solid Phase Peptide Synthesis, 2nd. ed., Pierce Chem. Co.: Rockford, Ill.; Kent, 1988, Ann. Rev. Biochem. 57: 957-89; and Gregg et al., 1990, Int. J. Peptide Protein Res. 55: 161-214 , which are incorporated herein by reference in their entirety.)

The use of solid phase methodology is preferred. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods or the like. Cycles of deprotection, neutralization and coupling of successive protected amino acid derivatives are used to link the amino acids from the C-terminus according to the amino acid sequence. For some synthetic peptides, an FMOC strategy using an acid-sensitive resin may be used. Preferred solid supports in this regard are divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenylaminomethyl)-phenoxymethyl resin, 2,4-dimethoxybenzhydryl-amine resin, and 4-(2′,4′-dimethoxyphenyl-FMOC-amino-methyl)-phenoxyacetamidonorleucyl-MBHA resin (Rink amide MBHA resin). In addition, acid-sensitive resins also provide C-terminal acids, if desired. A particularly preferred protecting group for alpha amino acids is base-labile 9-fluorenylmethoxy-carbonyl (FMOC).

Suitable protecting groups for the side chain functionalities of amino acids chemically compatible with BOC (t-butyloxycarbonyl) and FMOC groups are well known in the art. When using FMOC chemistry, the following protected amino acid derivatives are preferred: FMOC-Cys(Trit), FMOC-Ser(But), FMOC-Asn(Trit), FMOC-Leu, FMOC-Thr(Trit), FMOC-Val, FMOC-Gly, FMOC-Lys(Boc), FMOC-Gln(Trit), FMOC-Glu(OBut), FMOC-His(Trit), FMOC-Tyr(But), FMOC-Arg(PMC (2,2,5,7,8-pentamethylchroman-6-sulfonyl)), FMOC-Arg(BOC)2, FMOC-Pro, and FMOC-Trp(BOC). The amino acid residues can be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexa-fluorophosphate), PyBOP (benzotriazole-1-yl-oxy-tris-pyrrolidinophosphonium hexafluoro-phosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. Activation with HBTU (2-(1H-benzotriazole-1-yl),1,1,3,3-tetramethyluronium hexafluorophosphate) or HATU (2-(1H-7-aza-benzotriazole-1-yl), 1,1,3,3-tetramethyluronium hexafluoro-phosphate) in the presence of HOBt or HOAt (7-azahydroxybenztriazole) is preferred.

The solid phase method can be carried out manually, although automated synthesis on a commercially available peptide synthesizer (e.g., Applied Biosystems 431A or the like; Applied Biosystems, Foster City, Calif.) is preferred. In a typical synthesis, the first (C-terminal) amino acid is loaded on the chlorotrityl resin. Successive deprotection (with 20% piperidine/NMP (N-methylpyrrolidone)) and coupling cycles according to ABI FastMoc protocols (ABI user bulletins 32 and 33, Applied Biosystems) are used to build the whole peptide sequence. Double and triple coupling, with capping by acetic anhydride, may also be used.

The synthetic mimetic peptide is cleaved from the resin and deprotected by treatment with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, can be used. The peptide is separated from the resin by filtration and isolated by ether precipitation. Further purification may be achieved by conventional methods, such as gel filtration and reverse phase HPLC (high performance liquid chromatography). Synthetic calcitonin mimetics according to the present invention may be in the form of pharmaceutically acceptable salts, especially base-addition salts including salts of organic bases and inorganic bases. The base-addition salts of the acidic amino acid residues are prepared by treatment of the peptide with the appropriate base or inorganic base, according to procedures well known to those skilled in the art, or the desired salt may be obtained directly by lyophilization out of the appropriate base.

Generally, those skilled in the art will recognize that peptides as described herein may be modified by a variety of chemical techniques to produce compounds having essentially the same activity as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric and other organic salts, or may be converted to an amide. Thiols can be protected with any one of a number of well-recognized protecting groups, such as acetamide groups. Those skilled in the art will also recognize methods for introducing cyclic structures into the peptides of this invention so that the native binding configuration will be more nearly approximated. For example, a carboxyl terminal or amino terminal cysteine residue can be added to the peptide, so that when oxidized the peptide will contain a disulfide bond, thereby generating a cyclic peptide. Other peptide cyclizing methods include the formation of thioethers and carboxyl- and amino-terminal amides and esters.

Specifically, a variety of techniques are available for constructing peptide derivatives and analogs with the same or similar desired biological activity as the corresponding peptide compound but with more favorable activity than the peptide with respect to solubility, stability, and susceptibility to hydrolysis and proteolysis. Such derivatives and analogs include peptides modified at the N-terminal amino group, the C-terminal carboxyl group, and/or changing one or more of the amido linkages in the peptide to a non-amido linkage. It will be understood that two or more such modifications can be coupled in one peptide mimetic structure (e.g., modification at the C-terminal carboxyl group and inclusion of a —CH2- carbamate linkage between two amino acids in the peptide).

Amino terminus modifications include alkylating, acetylating, adding a carbobenzoyl group, and forming a succinimide group. Specifically, the N-terminal amino group can then be reacted to form an amide group of the formula RC(O)NH— where R is alkyl, preferably lower alkyl, and is added by reaction with an acid halide, RC(O)Cl or acid anhydride. Typically, the reaction can be conducted by contacting about equimolar or excess amounts (e.g., about 5 equivalents) of an acid halide to the peptide in an inert diluent (e.g., dichloromethane) preferably containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide for N-alkyl amide group of the formula RC(O)NR—. Alternatively, the amino terminus can be covalently linked to succinimide group by reaction with succinic anhydride. An approximately equimolar amount or an excess of succinic anhydride (e.g., about 5 equivalents) are used and the terminal amino group is converted to the succinimide by methods well known in the art including the use of an excess (e.g., ten equivalents) of a tertiary amine such as diisopropylethylamine in a suitable inert solvent (e.g., dichloromethane), as described in Wollenberg et al., U.S. Pat. No. 4,612,132, is incorporated herein by reference in its entirety. It will also be understood that the succinic group can be substituted with, for example, C2- through C6- alkyl or —SR substituents, which are prepared in a conventional manner to provide for substituted succinimide at the N-terminus of the peptide. Such alkyl substituents are prepared by reaction of a lower olefin (C2- through C6-alkyl) with maleic anhydride in the manner described by Wollenberg et al., supra., and —SR substituents are prepared by reaction of RSH with maleic anhydride where R is as defined above. In another advantageous embodiments, the amino terminus is derivatized to form a benzyloxycarbonyl-NH— or a substituted benzyloxycarbonyl-NH— group. This derivative is produced by reaction with approximately an equivalent amount or an excess of benzyloxycarbonyl chloride (CBZ-Cl) or a substituted CBZ-Cl in a suitable inert diluent (e.g., dichloromethane) preferably containing a tertiary amine to scavenge the acid generated during the reaction. In yet another derivative, the N-terminus comprises a sulfonamide group by reaction with an equivalent amount or an excess (e.g., 5 equivalents) of R—S(O)2Cl in a suitable inert diluent (dichloromethane) to convert the terminal amine into a sulfonamide, where R is alkyl and preferably lower alkyl. Preferably, the inert diluent contains excess tertiary amine (e.g., ten equivalents) such as diisopropylethylamine, to scavenge the acid generated during reaction. Reaction conditions are otherwise conventional (e.g., room temperature for 30 minutes). Carbamate groups are produced at the amino terminus by reaction with an equivalent amount or an excess (e.g., 5 equivalents) of R—OC(O)Cl or R—OC(O)OC6H4-p-NO2 in a suitable inert diluent (e.g., dichloromethane) to convert the terminal amine into a carbamate, where R is alkyl, preferably lower alkyl. Preferably, the inert diluent contains an excess (e.g., about 10 equivalents) of a tertiary amine, such as diisopropylethylamine, to scavenge any acid generated during reaction. Reaction conditions are otherwise conventional (e.g., room temperature for 30 minutes). Urea groups are formed at the amino terminus by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above preferably, the inert diluent contains an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group is replaced by an ester (e.g., —C(O)OR where R is alkyl and preferably lower alkyl), resins used to prepare the peptide acids are employed, and the side chain protected peptide is cleaved with base and the appropriate alcohol, e.g., methanol. Side chain protecting groups are then removed in the usual fashion by treatment with hydrogen fluoride to obtain the desired ester. In preparing peptide mimetics wherein the C-terminal carboxyl group is replaced by the amide —C(O)NR3R4, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH2). Alternatively, use of the chloromethylated resin during peptide synthesis coupled with reaction with ammonia to cleave the side chain Protected peptide from the support yields the free peptide amide and reaction with an alkylamine or a dialkylamine yields a side chain protected alkylamide or dialkylamide (i.e., the C-terminus is —C(O)NRR1, where R and R1 are alkyl and preferably lower alkyl). Side chain protection is then removed in the usual fashion by treatment with hydrogen fluoride to give the free amides, alkylamides, or dialkylamides.

In another alternative embodiment, the C-terminal carboxyl group or a C-terminal ester can be induced to cyclize by displacement of the —OH or the ester (—OR) of the carboxyl group or ester respectively with the N-terminal amino group to form a cyclic peptide. For example, after synthesis and cleavage to give the peptide acid, the free acid is converted in solution to an activated ester by an appropriate carboxyl group activator such as dicyclohexylcarbodiimide (DCC), for example, in methylene chloride (CH2Cl2), dimethyl formamide (DMF), or mixtures thereof. The cyclic peptide is then formed by displacement of the activated ester with the N-terminal amine. Cyclization, rather than polymerization, can be enhanced by use of very dilute solutions according to methods well known in the art.

Peptide mimetics as understood in the art and provided by the invention are structurally similar to the paradigm peptide of the invention, but have one or more peptide linkages optionally replaced by a linkage selected from the group consisting of: —CH2NH—, CH2S—, —CH2CH2-, —CH═CH— (in both cis and trans conformers), —COCH2-, CH(OH)CH2-, and —CH2SO—, by methods known in the art and further described in the following references: Spatola,1983, in chemistry and biochemistry of amino acids, peptides, and proteins, (Weinstein, ed.), Marcel Dekker: New York, p. 267; Spatola, 1983, Peptide Backbone Modifications 1: 3; Morley, 1980, Trends Pharm. Sci. pp. 463-468; Hudson et al., 1979, Int. J. Pept. Prot. Res. 14: 177-185; Spatola et al., 1986, Life Sci. 38: 1243-1249; Hann, 1982, J. Chem. Soc. Perkin Trans. I 307-314; Almquist et al., 1980, J. Med. Chem. 23: 1392-1398; Jennings-White et al., 1982, Tetrahedron Lett. 23: 2533; Szelke et al., 1982, European Patent Application, Publication No. EP045665A; Holladay et al., 1983, Tetrahedron Lett. 24: 4401-4404; and Hruby, 1982, Life Sci. 31: 189-199, each of which is incorporated herein by reference. Such peptide mimetics may have significant advantages over polypeptide embodiments, including, for example: being more economical to produce, having greater chemical stability or enhanced pharmacological properties (such half-life, absorption, potency, efficacy, etc.), reduced antigenicity, and other properties.

Mimetic analogs of the peptides of the invention may also be obtained using the principles of conventional or rational drug design (see, Andrews et al., 1990, Proc. Alfred Benzon Symp. 28: 145-165; McPherson, 1990, Eur. J. Biochem. 189:1-24; Hol et al., 1989a, in Molecular Recognition: Chemical and Biochemical Problems, (Roberts, ed.); Royal Society of Chemistry; pp. 84-93; Hol, 1989b, Arzneim-Forsch. 39:1016-1018; Hol, 1986, Agnew Chem. Int. Ed. Engl. 25: 767-778, the disclosures of which are herein incorporated by reference).

In accordance with the methods of conventional drug design, the desired mimetic molecules are obtained by randomly testing molecules whose structures have an attribute in common with the structure of a “native” peptide. The quantitative contribution that results from a change in a particular group of a binding molecule can be determined by measuring the biological activity of the putative mimetic in comparison with the activity of the peptide. In a preferred embodiment of rational drug design, the mimetic is designed to share an attribute of the most stable three-dimensional conformation of the peptide. Thus, for example, the mimetic may be designed to possess chemical groups that are oriented in a way sufficient to cause ionic, hydrophobic, or van der Waals interactions that are similar to those exhibited by the peptides of the invention, as disclosed herein.

The preferred method for performing rational mimetic design employs a computer system capable of forming a representation of the three-dimensional structure of the peptide, such as those exemplified by Hol, 1989a, ibid.; Hol, 1989b, ibid.; and Hol, 1986, ibid. Molecular structures of the peptido-, organo- and chemical mimetics of the peptides of the invention are produced according to those with skill in the art using computer-assisted design programs commercially available in the art. Examples of such programs include sybyl 6.5®, hqsar™, and alchemy 2000™ (Tripos); galaxy™ and am2000™ (AM Technologies, Inc., San Antonio, Tex.); catalyst™ and cerius™ (Molecular Simulations, Inc., San Diego, Calif.); cache products™, tsar™, amber™, and chem-x™ (Oxford Molecular Products, Oxford, Calif.) and chembuilder3d™ (Interactive Simulations, Inc., San Diego, Calif.).

The peptido-, organo- and chemical mimetics produced using the peptides disclosed herein using, for example, art-recognized molecular modeling programs are produced using conventional chemical synthetic techniques, most preferably designed to accommodate high throughput screening, including combinatorial chemistry methods. Combinatorial methods useful in the production of the peptido-, organo- and chemical mimetics of the invention include phage display arrays, solid-phase synthesis and combinatorial chemistry arrays, as provided, for example, by SIDDCO, Tuscon, Ariz.; Tripos, Inc.; Calbiochem/Novabiochem, San Diego, Calif.; Symyx Technologies, Inc., Santa Clara, Calif.; Medichem Research, Inc., Lemont, Ill.; Pharm-Eco Laboratories, Inc., Bethlehem, Pa.; or N.V. Organon, Oss, Netherlands. Combinatorial chemistry production of the peptido-, organo- and chemical mimetics of the invention are produced according to methods known in the art, including but not limited to techniques disclosed in Terrett, 1998, combinatorial chemistry, Oxford University Press, London; Gallop et al., 1994, “Applications of combinatorial technologies to drug discovery. 1. Background and peptide combinatorial libraries,” J. Med. Chem. 37: 1233-51; Gordon et al., 1994, “Applications of combinatorial technologies to drug discovery. 2. Combinatorial organic synthesis, library screening strategies, and future directions,” J. Med. Chem. 37: 1385-1401; Look et al., 1996, Bioorg. Med. Chem. Lett. 6: 707-12; Ruhland et al., 1996, J. Amer. Chem. Soc. 118: 253-4; Gordon et al., 1996, Acc.Chem. Res. 29: 144-54; Thompson & Ellman, 1996, Chem. Rev. 96: 555-600; Fruchtel & Jung, 1996, Angew. Chem. Int. Ed. Engl. 35: 17-42; Pavia, 1995, “The Chemical Generation of Molecular Diversity”, Network Science Center, www.netsci.org; Adnan et al., 1995, “Solid Support Combinatorial Chemistry in Lead Discovery and SAR Optimization,” Id., Davies and Briant, 1995, “Combinatorial Chemistry Library Design using Pharmacophore Diversity,” Id., Pavia, 1996, “Chemically Generated Screening Libraries: Present and Future,” Id.; and U.S. Pat. Nos. 5,880,972 to Horlbeck; 5,463,564 to Agrafiotis et al.; 5,331573 to Balaji et al.; and 5,573,905 to Lerner et al.

The newly synthesized polypeptides can be substantially purified by preparative high performance liquid chromatography (see, for example, Creighton, PROTEINS: STRUCTURES AND MOLECULAR PRINCIPLES, WH Freeman and Co., New York, N.Y., 1983). The composition of a synthetic polypeptide of the present invention can be confirmed by amino acid analysis or sequencing by, for example, the Edman degradation procedure (see, Creighton, supra). Additionally, any portion of the amino acid sequence of the polypeptide can be altered during direct synthesis and/or combined using chemical methods with sequences from other proteins to produce a variant polypeptide or a fusion polypeptide.

Assessment of Therapeutic Utility of Human Antagonist

To assess the potential efficacy of a particular antagonist in cancer therapy, the antagonist can be tested in vitro in cell proliferation assays as detailed in Examples 6. In addition, the effect on plasma half-life of coupling the VPAC1 selective antagonist to a non-protein polymer can be measured in vivo with a rat pharmacokinetic study according to Example 7.

Pharmaceutical Compositions

Any of the VPAC1 selective antagonists described above can be provided in a pharmaceutical composition comprising a pharmaceutically acceptable carrier. The pharmaceutically acceptable carrier preferably is non-pyrogenic. The compositions can be administered alone or in combination with at least one other agent, such as stabilizing compound, which can be administered in any sterile, biocompatible pharmaceutical carrier, including, but not limited to, saline, buffered saline, dextrose, and water. A variety of aqueous carriers may be employed, e.g., 0.4% saline, 0.3% glycine, and the like. These solutions are sterile and generally free of particulate matter. These solutions may be sterilized by conventional, well-known sterilization techniques (e.g., filtration).

The compositions may contain pharmaceutically acceptable auxiliary substances as required. Acceptable auxiliary substances preferably are nontoxic to recipients at the dosages and concentrations employed. The pharmaceutical composition can contain auxiliary substances for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See Remington's Pharmaceutical Sciences (18th Ed., A. R. Gennaro, ed., Mack Publishing Company 1990).

The concentration of the antagonist of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected. If desired, more than one type of antagonist, for example with different Kd for VPAC1 binding, can be included in a pharmaceutical composition.

The compositions can be administered to a patient alone, or in combination with other agents, drugs or hormones. In addition to the active ingredients, these pharmaceutical compositions can contain suitable pharmaceutically acceptable carriers comprising excipients and auxiliaries that facilitate processing of the active compounds into preparations which can be used pharmaceutically.

Acceptable formulation materials preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See Remington's Pharmaceutical Sciences (18th Ed., A. R. Gennaro, ed., Mack Publishing Company 1990.

The optimal pharmaceutical composition can be determined by a skilled artisan depending upon, for example, the intended route of administration, delivery format, and desired dosage. (See, e.g., Remington's Pharmaceutical Sciences, supra). Such compositions can influence the physical state, stability, rate of in vivo release, and rate of in vivo clearance of the nucleic acid molecule or bone density modulator of the invention.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the invention, pharmaceutical compositions of the invention can be prepared for storage by mixing the selected composition having the desired degree of purity with optional formulation agents (Remington's Pharmaceutical Sciences, supra) in the form of a lyophilized cake or an aqueous solution. Further, the composition can be formulated as a lyophilizate using appropriate excipients such as sucrose.

The pharmaceutical compositions can be selected for parenteral delivery. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in the invention can be in the form of a pyrogen-free, parenterally acceptable, aqueous solution comprising the desired molecule of the invention in a pharmaceutically acceptable vehicle. A particularly suitable vehicle for parenteral injection is sterile distilled water in which the molecule is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which may then be delivered via a depot injection. Hyaluronic acid can also be used, which can have the effect of promoting sustained duration in the circulation. Other suitable means for the introduction of the desired molecule include implantable drug delivery devices.

In one embodiment, a pharmaceutical composition can be formulated for inhalation. For example, a nucleic acid molecule or bone density modulator of the invention can be formulated as a dry powder for inhalation. Inhalation solutions can also be formulated with a propellant for aerosol delivery. In yet another embodiment, solutions can be nebulized. Pulmonary administration is further described in PCT Pub. No. WO 94/20069, which describes the pulmonary delivery of chemically modified proteins.

In other embodiments, certain formulations can be administered orally. In one embodiment of the invention, nucleic acid molecules or bone density modulators of the invention that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the molecule or modulator of the invention. Diluents, flavorings, low melting point waxes, vegetable oils, lubricants, suspending agents, tablet disintegrating agents, and binders may also be employed.

Another pharmaceutical composition can involve an effective quantity of nucleic acid molecules or bone density modulators of the invention in a mixture with non-toxic excipients that are suitable for the manufacture of tablets. By dissolving the tablets in sterile water, or another appropriate vehicle, solutions can be prepared in unit-dose form.

Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919 and European Patent No. 058481), copolymers of L-glutamic acid and gamma ethyl-L-glutamate (Sidman et al., 1983, Biopolymers 22:547-56), poly(2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., supra) or poly-D(−)-3-hydroxybutyric acid (European Patent No. 133988). Sustained-release compositions may also include liposomes, which can be prepared by any of several methods known in the art. See, e.g., Eppstein et al., 1985, Proc. Natl. Acad. Sci. USA 82:3688-92; and European Patent Nos. 036676, 088046, and 143949.

A pharmaceutical composition to be used for in vivo administration typically must be sterile. This may be accomplished by filtration through sterile filtration membranes. Where the composition is lyophilized, sterilization using this method may be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Pharmaceutical compositions of the invention can be administered by any number of routes as described herein including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means.

After pharmaceutical compositions have been prepared, they can be placed in an appropriate container and labeled for treatment of an indicated condition. Such labeling would include amount, frequency, and method of administration.

Therapeutic Methods

The present invention provides methods of ameliorating symptoms of a disorder by binding the VPAC1, and inhibiting VPAC1-mediated activity such as cell proliferation. These disorders include, but are not limited to the various forms of cancer.

In one embodiment of the invention, a therapeutically effective dose of a VPAC1 selective antagonist of the invention and/or a pharmaceutical composition of the invention is administered to a patient having a disorder characterized by elevated VPAC1 expression such as those disorders above.

Determination of a Therapeutically Effective Dose

The determination of a therapeutically effective dose is well within the capability of those skilled in the art. A therapeutically effective dose refers to the amount of antagonist that is used to effectively treat asthma compared with the efficacy that is evident in the absence of the therapeutically effective dose.

The therapeutically effective dose can be estimated initially in animal models, usually rats, mice, rabbits, dogs, pigs or non-human primates. The animal model also can be used to determine the appropriate concentration range and route of administration. Such information can then be used to determine useful doses and routes for administration in humans.

Therapeutic efficacy and toxicity, e.g., ED50 (the dose therapeutically effective in 50% of the population) and LD50 (the dose lethal to 50% of the population) of a human antagonist, can be determined by standard pharmaceutical procedures in cell cultures or experimental animals. The dose ratio of toxic to therapeutic effects is the therapeutic index, and it can be expressed as the ratio, LD50/ED50.

Pharmaceutical compositions that exhibit large therapeutic indices are preferred. The data obtained from animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the patient who requires treatment. Dosage and administration are adjusted to provide sufficient levels of the antagonist or to maintain the desired effect. Factors that can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Effective in vivo dosages of an antagonist are in the range of about 5 μg to about 50 μg/kg, about 50 μg to about 5 mg/kg, about 100 μg to about 500 μg/kg of patient body weight, and about 200 to about 250 μg/kg of patient body weight.

The mode of administration of VPAC1 selective antagonist-containing pharmaceutical compositions of the invention can be any suitable route which delivers the antagonist to the host. Pharmaceutical compositions of the invention are particularly useful for parenteral administration, i.e., subcutaneous, intramuscular, intravenous, intracheal or intranasal and other modes of pulmonary administration.

All patents and patent applications cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLES

### Example 1

### Peptide Synthesis Methodology

The following general procedure was followed to synthesize the polypeptides of the invention. Peptide synthesis was carried out by the FMOC/t-Butyl strategy (Peptide Synthesis Protocols (1994), Volume 35 by Michael W. Pennington & Ben M. Dunn) under continuous flow conditions using Rapp-Polymere PEG-Polystyrene resins (Rapp-Polymere, Tubingen, Germany). At the completion of synthesis, peptides are cleaved from the resin and de-protected using TFA/DTT/H2O/Triisopropyl silane (88/5/5/2). Peptides were precipitated from the cleavage cocktail using cold diethyl ether. The precipitate was washed three times with the cold ether and then dissolved in 5% acetic acid prior to lyophilization. Peptides were checked by reversed phase chromatography on a YMC-Pack ODS-AQ column (YMC, Inc., Wilmington, N.C.) on a Waters ALLIANCE® system (Waters Corporation, Milford, Mass.) using water/acetonitrile with 3% TFA as a gradient from 0% to 100% acetonitrile, and by MALDI mass spectrometry on a VOYAGER DE™ MALDI Mass Spectrometer, (model 5-2386-00, PerSeptive BioSystems, Framingham, Mass.). Those peptides not meeting the purity criteria of >95% are purified by reversed phase chromatography on a Waters Delta Prep 4000 HPLC system (Waters Corporation, Milford, Mass.).

### Example 2

### Peptide Pegylation

Site-specific introduction of PEG was effected by introducing a unique cysteine mutation at the C-terminal peptide followed by PEGylating the cysteine via a stable thioether linkage between the sulfhydryl of the peptide and maleimide group of the methoxy-PEG-maleimide reagent (Inhale/Shearwater). A 2-fold molar excess of mPEG-mal (MW 22 kD or 43 kD) reagent was added to 1 mg of peptide dissolved in reaction buffer at pH 6 (0.1 M Na phosphate/ 0.1M NaCl/ 0.1M EDTA). After 0.5 hour at room temperature, the reaction was stopped with 2-fold molar excess of DTT to mPEG-mal. The peptide-PEG-mal reaction mixture was applied to a cation exchange column to remove residual PEG reagents followed by gel filtration column to remove residual free peptide. The purity, mass, and number of PEGylated sites were determined by SDS-PAGE and MALDI-TOF mass spectrometry.

### Example 3

### VPAC1 and VPAC2 Transfected CHO Cell Lines

In order to test for selective binding of the VPAC1 selective antagonist to the VPAC1, both the VPAC1 and VPAC2 receptors were expressed in CHO cells using the following procedure. The human VPAC1 and the VPAC2 were cloned via RT PCR from human heart mRNA and human testis mRNA, respectively, using TaqPlus Precision PCR System (Stratagene). The PCR products were subcloned into pCDNA3.1 (Invitrogen) for in vitro translation and mammalian expression. The cell line chosen for expression was the CHOcreluc line already expressing a cAMP response element-luciferase reporter along with Gα16. These cells were grown under hygromycin selection at 0.4 mg/ml. On the day of transfection, CHOcreluc cells at 70% confluency were washed with serum free media and transfected using Lipofectamine Plus Reagent (Gibco BRL). Stable pools were selected in the presence of 0.4 mg/ml hygromycin and 1.5 mg/ml G418. Once viably-frozen stocks had been made from these pools they were cloned by limiting dilution. Expression and functionality of the receptors were confirmed by treatment of the cells with PACAP27 and VIP peptides and luciferase assay.

### Example 4

### Receptor Competition Assay

The capacity of the VPAC1 selective antagonist to selectively bind the VPAC1 as opposed to the VPAC2 receptor was measured using membranes prepared from CHO cells transfected with both receptors as described in Example 3. Cells were washed with phosphate buffered saline (PBS), scraped in homogenization buffer (10 mM Tris pH 7.4, 2 mM EDTA, 5 mM MgCl2, 1 mM PMSF), followed by centrifugation at 4000 g for 10 minutes at 4° C. The cell pellet was resuspended in homogenization buffer and homogenized using a Polytron. Membranes were collected by centrifugation at 30,000 g for 30 minutes at 4° C., resuspended in homogenization buffer, and stored at −80° C. until use. To measure binding of PACAP peptides, 10 ug membrane was incubated with 0.1 nM 1251-PACAP27 (NEN) in the presence of increasing concentrations of peptide, in a total volume of 100 μl 20 mM Hepes (pH 7.4), 150 mM NaCl, 0.5% BSA, 2 mM MgCl2, and 0.1 mg/ml bacitracin. After incubating at 37° C. for 20 minutes, bound ligand was collected on GF/C filters pretreated with 0.1% polyethylenimine. The filters were washed with cold 25 mM NaPO4 containing 1% BSA and counted in a gamma counter. All reagents were purchased from Sigma unless otherwise indicated.

R2P16 (SEQ ID NO 6) and PEGylated R2P3 (SEQ ID NO 4) and PEGylated R2P11 (SEQ ID NO 5) demonstrated 200-700-fold lower IC50 values for PACAP27 binding to VPAC1 than to VPAC2. See Table 3. These data demonstrate that the peptides of the invention selectively antagonize binding to the VPAC1.

### Example 5

### Cyclic AMP SPA.

The ability of the VPAC1 selective antagonist to selectively antagonize VPAC1 mediated cellular activity was assessed by measuring the concentration of cyclic AMP in cell extracts following exposure of the cells to VIP with and without the VPAC1 selective antagonist present. CHO cells expressing the VPAC1 or VPAC2 were plated in 96-well plates (Costar) at 8×104 cell/well and grown at 37° C. for 24 hours in a MEM+nucleosides+glutamine (Gibco BRL), 10% FBS, 100 μg/ml Pen/Strep, 0.3 mg/ml glutamine, 1 mM HEPES, 0.5 mg/ml Geneticin (Gibco BRL). The medium was removed and the plates were washed with PBS. The cells were incubated in Hepes-PBS-BSA with 0.4 mg/ml Soybean Trypsin Inhibitor, 0.5 mg/ml Bacitracin, 100 uM IBMX, for 15 minutes at 37° C. Following equilibration at 37° C. in a 5% CO2/95% O2 environment for 10 min, increasing amounts of peptide antagonist were added to the cells followed immediately by 1 nM VIP for 15 minutes. Cyclic AMP in the cell extracts was quantitated using the cAMP SPA direct screening assay system (Amersham Pharmacia Biotech Inc, Piscataway, N.J.). The EC50 of VIP (VIP concentration at which 50% of maximum activity is achieved) for VPAC1 was determined to be 0.3 nM and at 1 nM VIP the maximum activity has already been achieved. Thus, 1 nM was chosen as the VIP concentration to be competed by the peptide antagonists. R2P16 and PEGylated R2P3 and R2P11 demonstrated IC50 values for VIP binding to VPAC1 in the range of 50 to 181 nM. See Table 3. These data demonstrate that the peptides of the invention effectively antagonize VIP mediated cAMP generation via the VPAC1.

### Example 6

### NCI-H727 Cancer Cell Proliferation Assay

This example demonstrates how peptides of the invention are capable of inhibiting the proliferation of cancer cell lines. NCI-H727 is an adherent human non-small cell lung carcinoma cell line. The cells are grown in RPMI-1640 plus 2 mM L-Glutamine and 10% FBS and cells subcultured in the following manner: Medium was removed and cells were rinsed once in PBS solution. To harvest the cells 10 mls PBS containing 2 ml of trypsin-EDTA solution was added to a 75 ml flask. The flask was incubated at 37° C. until the cells detached. Fresh culture medium was added, aspirated and dispensed into new culture flasks. A split ratio of 1:3 to 1:4 was carried out 2 times per week. The 96-well assay was performed as follows: Day 1) Cells were seeded at 7000 cells/well in 0.2 ml complete medium/well and incubated overnight at 37° C. Day 2) Complete medium was aspirated from wells and 200 ul/well PBS was added and aspirated. Cells were then treated with peptides using the assay medium RPMI-1640 plus 2 mM L-Glutamine and 0.2% FBS at a final volume of 200 ul/well and incubated for 2 days. Day 4) Alamar Blue (10% of total volume) was added to wells and absorbance (530/590 nm) read at 0, 4, 6, 8, 10 and 24 hours. Peptide R2P16 was found to have an IC50 three times lower than that of the reference compound R2P2. See Table 3. These data demonstrate that R2P16 antagonizes the VPAC1's ability to promote cell proliferation in a disease relevant assay.

### Example 7

### Rat Pharmacokinetic Study

Adult male Sprague-Dawley rats weighing 250 to 300 grams will be cannulated with jugular vein catheter for blood sample collection. In addition, the rats in the intravenous (IV) dose group can be cannulated with femoral vein catheters for drug administration.

The rats will be given either VPAC1 selective antagonist or a PEGylated VPAC1 selective antagonist at doses of 1 and 0.5 mg/kg, respectively. Both IV and SC (subcutaneous) routes of administration will be used. The IV dose can be given by injection directly into the indwelling femoral vein catheter while the SC dose is given by injection into the dorsal thoracic region. Three rats will be used for each dose group.

Following a single bolus injection (IV or SC), blood samples will be collected at predose and at predetermined times up to 168 hours post-dose. Centrifugation for samples will be scheduled within 1 hour of collection and plasma harvested and placed on dry ice prior to storage at approximately −70° C.

Plasma concentrations of VPAC1 selective antagonist or a PEGylated VPAC1 selective antagonist can be quantified with an enzyme-linked immunoassay in which anti-VPAC1RA antibody will be used as a coating and detection reagent. The lower limit of quantification for this assay is 0.2 ng/ml. Pharmacokinetic parameters can be generally derived by non-compartmental analysis using WinNonlin (Pharsight, Mountain view, Calif.). Of particular interest will be the assessment of absorption and elimination kinetics, distribution volumes as well as the amount absorbed.

TABLE 2
POLYPEPTIDE SEQUENCES

| | | |
|---|---|---|
| Seq. ID No. | Name | Sequence |

| | | |
|---|---|---|
| 1 | Vasoactive Intestinal | HSDAVFTDNYTRLRKQMAVKKYLNSILN* |
| | Peptide | |
| 2 | Growth Hormone | YADAIFTNSYRKVLGQLSARKLLQDIMSR* |
| | Releasing Hormone | |
| 3 | PACAP27 | HSDGIFTDSYSRYRKQMAVKKYLAAVL* |
| 4 | R2P3 | HfDAVFTNSYRKVLKRLSARKLLQDILC* |
| 5 | R2P11 | HfDAVFTNSYRKVLKRLSVRKLLQDILC* |
| 6 | R2P16 | HfDAVFTNSYRKVLKRLSARKLLQSIL* |

H = N-terminal acylated histidine.
f = D-Phe.
* = C-terminal amidation.

Underlined amino acids represent non-conservative mutations from VIP.

TABLE 3
VPAC1 SELECTIVE ANTAGONIST BINDING AND
CELL-BASED ACTIVITY

| | CHO Receptor | | | | |
|---|---|---|---|---|---|
| | Competition Binding | | | CHO | H727 |
| | VPAC1 | | | VPAC1 | Prolif. |
| | Binding | VPAC2 | VPAC2/1 | cAMP | Inhibition |
| Peptide | (IC 50) | Binding | Selectivity | inhibition | (×103) |
| R2P2 | 31 + 15 | >10000 | >300 | 26 + 6 | 27 ± 4.0 |
| R2P16 | 17 + 6 | >10000 | >700 | 50 + 12 | 8.0 ± 0.1 |
| R2P3- | 21 + 4 | >10000 | >480 | 181 + 44 | |
| PEG22kD | | | | | |
| R2P11- | 49 + 2 | >10000 | >200 | 101 + 24 | |
| PEG22kD | | | | | |

1. A purified hybrid polypeptide sequence, identified as Seq. ID NO. 6, comprising a vasoactive intestinal peptide component and a growth hormone releasing hormone component, wherein said hybrid sequence is capable of selectively binding to and antagonizing a cellular VPAC1 receptor at significantly lower concentrations than those concentrations at which it binds to and antagonizes a cellular VPAC2 receptor.

2. The polypeptide sequence of claim 1 wherein said sequence selectively inhibits the binding of PACAP27 to cell membranes expressing the VPAC1 with an IC50 of about 0.1 nM to about 10 μM.

3. The polypeptide sequence of claim 1 wherein said sequence selectively inhibits the binding of PACAP27 to cell membranes expressing the VPAC1 with an IC50 of about 0.5 nM to about 1 μM.

4. The polypeptide sequence of claim 1 wherein said sequence selectively inhibits the binding of PACAP27 to cell membranes expressing the VPAC1 with an IC50 of about 1.0 nM to about 100 nM

5. The polypeptide sequence of claim 1 wherein said sequence inhibits the VIP-mediated generation of cAMP with an IC50 of about 0.1 nM to about 10 μM.

6. The polypeptide sequence of claim 1 wherein said sequence inhibits the VIP-mediated generation of cAMP with an IC50 of about 0.5 nM to about 1 μM.

7. The polypeptide sequence of claim 1 wherein said sequence inhibits the VIP-mediated generation of cAMP with an IC50 of about 1.0 nM to about 100 nM.

8. The polypeptide sequence of claim 1 wherein said sequence inhibits the proliferation of H727 cells with an IC50 of about 0.1 nM to about 10 μM.

9. The polypeptide sequence of claim 1 wherein said sequence inhibits the proliferation of H727 cells with an IC50 of about 0.5 nM to about 1 μM.

10. The polypeptide sequence of claim 1 wherein said sequence inhibits the proliferation of H727 cells with an IC50 of about 1.0 nM to about 100 nM.

11. A method of treating a human disorder in which the purified VPAC1 is overexpressed, comprising the steps of:
a) providing a human having a condition in which VPAC1 is expressed in certain cells; and
b) administering to said human an effective amount of a purified VPAC1 antagonist until said human condition is ameliorated.

12. A purified hybrid polypeptide sequence selected from the group consisting of SEQ ID NOs. 4 and 5, coupled to a non-protein polymer selected from the group consisting of polyethylene glycol, polypropylene glycol and polyoxyalkylenes wherein said sequence comprises a vasoactive intestinal peptide component and a growth hormone releasing hormone component, and wherein said hybrid polypeptide sequence selectively binds to and antagonizes VPAC1 receptor at significantly lower concentrations than those concentrations at which it binds to and antagonizes VPAC2 receptor.

13. The polypeptide sequence of claim 12, wherein said polypeptide selectively inhibits the binding of PACAP27 to cells expressing the VPAC1 with an IC50 of about 0.1 nM to about 10 μM.

14. The polypeptide sequence of claim 12, wherein said polypeptide selectively inhibits the binding of PACAP27 to cells expressing the VPAC1 with an IC50 of about 0.5 nM to about 1 μM.

15. The polypeptide sequence of claim 12, wherein said polypeptide selectively inhibits the binding of PACAP27 to cells expressing the VPAC1 with an IC50 of about 1.0 nM to about 100 nM.

16. The polypeptide sequence of claim 12, wherein said polypeptide selectively inhibits VIP-mediated generation of cAMP with an IC50 of about 0.1 nM to about 10 μM.

17. The polypeptide sequence of claim 12, wherein said polypeptide selectively inhibits VIP-mediated generation of cAMP with an IC50 of about 0.5 nM to about 1 μM.

18. The polypeptide sequence of claim 12, wherein said polypeptide selectively inhibits VIP-mediated generation of cAMP with an IC50 of about 1.0 nM to about 100 nM.
